(19) United States
(12) Patent Application Publication
(10) Pub. No.: US 2014/0088516 A1
(43) Pub. Date: Mar. 27, 2014

(54) VISUALLY SIZE-GAUGING HOLLOW NEEDLE ASSEMBLY

(71) Applicant: Jon R. Stevenson, Powell, OH (US)

(72) Inventor: Jon R. Stevenson, Powell, OH (US)

(21) Appl. No.: 14/090,544

(22) Filed: Nov. 26, 2013

Related U.S. Application Data
(63) Continuation-in-part of application No. 13/343,284, filed on Jan. 4, 2012, now Pat. No. 8,617,111.
(60) Provisional application No. 61/429,528, filed on Jan. 4, 2011.

(51) Int. Cl. A61M 5/00

(52) U.S. Cl. CPC A61M 5/00
USPC 604/263; 604/272

(57) ABSTRACT

A visually size-gauging hollow needle assembly having a needle, stylet, and hub. At least one of a needle sleeve, needle bedding, needle attachment compound, or external indicia is size-gauging, generally by color, to indicate different sized (gauging) needles. In some embodiments, a hub sidewall has at least one external surface with at least one humanly tactilely discernible surface feature indicating the rotational relationship between the hub and the needle. An operator pinching the hub between a thumb and forefinger thus immediately feels these surface features, which may reflect the position of a needle barrel. When the assembly is rotated between the thumb and forefinger, these orientation features rotate as well, and the difference in tactile sensation immediately alerts the operator to the position of the needle bevel.

### CROSS-REFERENCE TO RELATED APPLICATIONS

This application claims the benefit of U.S. Provisional Patent Application Ser. No. 61/429,528; filed on Jan. 4, 2011, and U.S. Utility patent application Ser. No. 13/343,284, filed Jan. 4, 2012; all of which are incorporated by reference as if completely written herein.

### TECHNICAL FIELD

The present invention relates generally to the field of hollow procedural needles, and in particular to a hollow procedural needle bearing visually perceptible indicia indicating the gauge of the needle in a novel manner. In particular, certain embodiments include a color-indexed needle sleeve visible through an at least partially visually clear needle hub.

### BACKGROUND OF THE INVENTION

In general, procedural needles are hollow needles typically used for a wide variety of medical procedures, and in some, but not all applications, are often traditionally referred to as “hypodermic” or “spinal” needles. Traditional procedural needles share a common trait. They generally have an exit port at or near the injection end of the needle, and this port may be at the needle end, or side, or most commonly, is central to a needle bevel, which provides a sharp point and makes the needle easier to push through skin or other tissues.

In needles having an exit port on the side, or as part of a bevel, therefore, there is a rotational orientation to the needle. The exit port, and/or bevel, may be directed in any direction around a 360 degree arc—and once the tip of the needle is inserted below the skin, it become very difficult to determine in which direction the exit port and/or bevel is pointing. There may be an index tab in the hub of the needle, often engaging a tab on the needle stylet, but this slot and tab arrangement is very small, and very difficult to feel.

Most typically, needles are inserted by having the operator grasp the needle hub in a pinching action between the thumb and forefinger, and what has long been needed is a hollow needle assembly in which a humanly tactile discernible feature or features on the hollow needle assembly itself gives the operator the information, continuously and without needing to visually check, the position of a hidden bevel of a partially inserted needle.

In particular, procedural needles, while they are supplied in packaging that carries important information about the needle, particularly the size (gauge) of the needle; needles are often themselves made with indicia bearing information. In particular, it is not unusual to have the stylets or stylet hubs of needles follow a color-coded scheme that allows for visual differentiation among needles of different gauge. However, when the stylet is removed, and the needle alone is visualized, the loss of the color-coded stylet or stylet hub makes it very difficult to differentiate among different needles. This is particularly so, since the hubs of hollow procedural needles are typically made of clear materials, so that any fluid aspirated into the hub is more easily identifiable. Such clear materials present a great challenge to any visual size-gauging scheme.

### BRIEF DESCRIPTION OF THE DRAWINGS

Without limiting the scope of the present invention as claimed below and referring now to the drawings and figures:

FIG. 1 is an elevated perspective view of a portion of an embodiment of the instant invention;

FIG. 2 is a side view of a portion of the embodiment of FIG. 1;

FIG. 3 is a side view of a portion of the embodiment of FIG. 1;

FIG. 4 is a top view of a portion of the embodiment of FIG. 1;

FIG. 5 is a top view of a portion of the embodiment of FIG. 1;

FIG. 6 is a partially exploded side view of a portion of the embodiment of FIG. 1;

FIG. 7 is a side view of a portion of the embodiment of FIG. 1;

FIG. 8 is a partially exploded top view of a portion of the embodiment of FIG. 1;

FIG. 9 is a top view of a portion of the embodiment of FIG. 1;

FIG. 10 is an elevated perspective view of a portion of the embodiment of FIG. 1;

FIGS. 11A-C are a series of partially exploded views showing partial assembly methods of an embodiment of FIG. 1;

FIG. 12 is a section view taken along line 12-12 of FIG. 11;

FIG. 13 is an elevated perspective view of an embodiment of the instant invention, showing a detachable wing hub;

FIG. 14 is a top plan view of an embodiment of the instant invention, partially disassembled, showing details of the stylet and hub; and

FIG. 15 is a top plan view of an embodiment of the instant invention, assembled, showing details of the stylet and hub.

These drawings are provided to assist in the understanding of the exemplary embodiments of the invention as described in more detail below and should not be construed as unduly limiting the invention. In particular, the relative spacing, positioning, sizing and dimensions of the various elements illustrated in the drawings are not drawn to scale and may have been exaggerated, reduced or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

The present invention includes a rotational orientation indicating hollow needle assembly, as well as embodiments directed to a visually size-gauging hollow needle assembly. The invention enables a significant advance in the state of the art. The preferred embodiments of the apparatus accomplish this by new and novel methods that are configured in unique and novel ways and which demonstrate previously unavailable but preferred and desirable capabilities. The description set forth below in connection with the drawings is intended merely as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention.

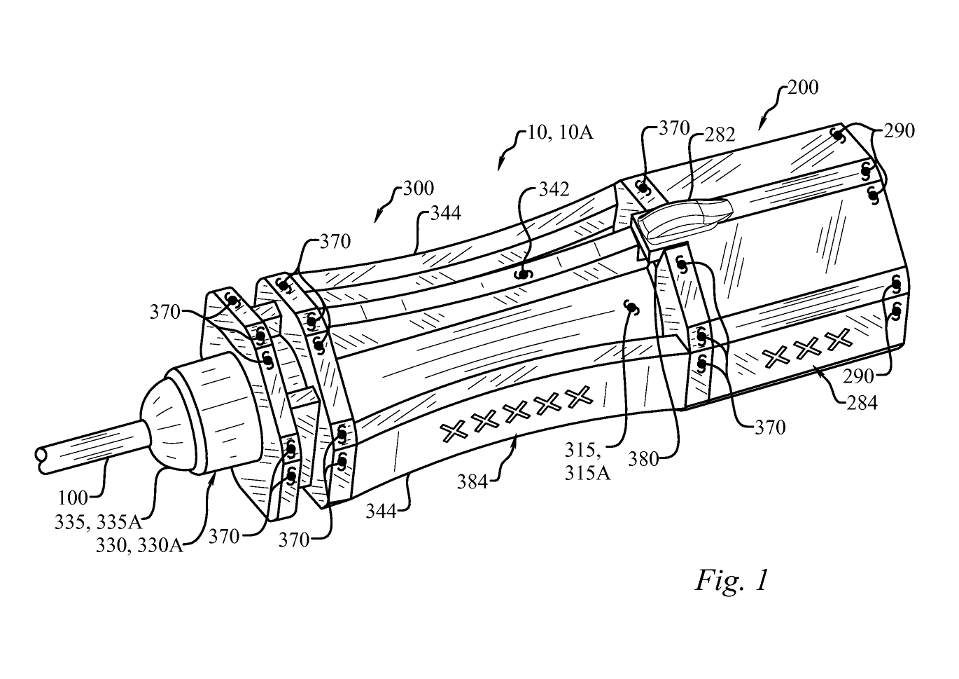

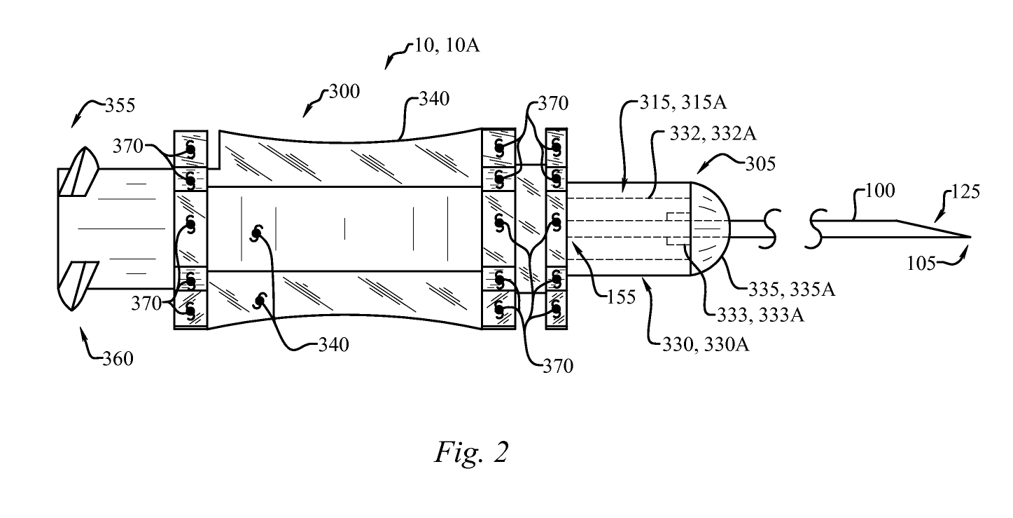

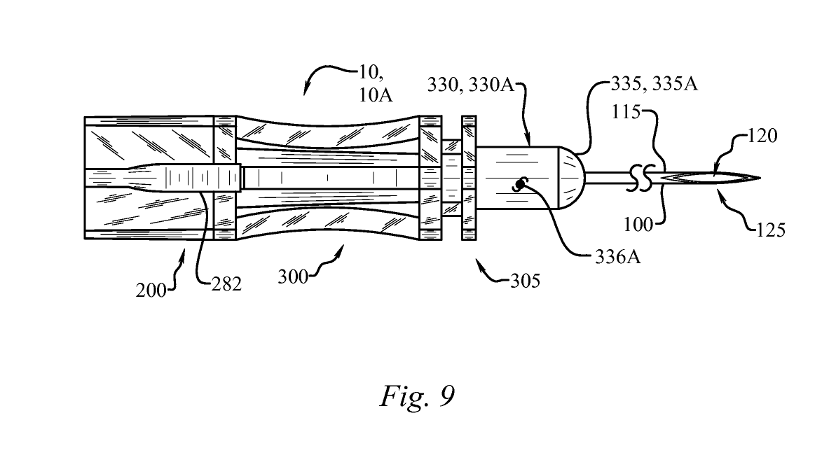

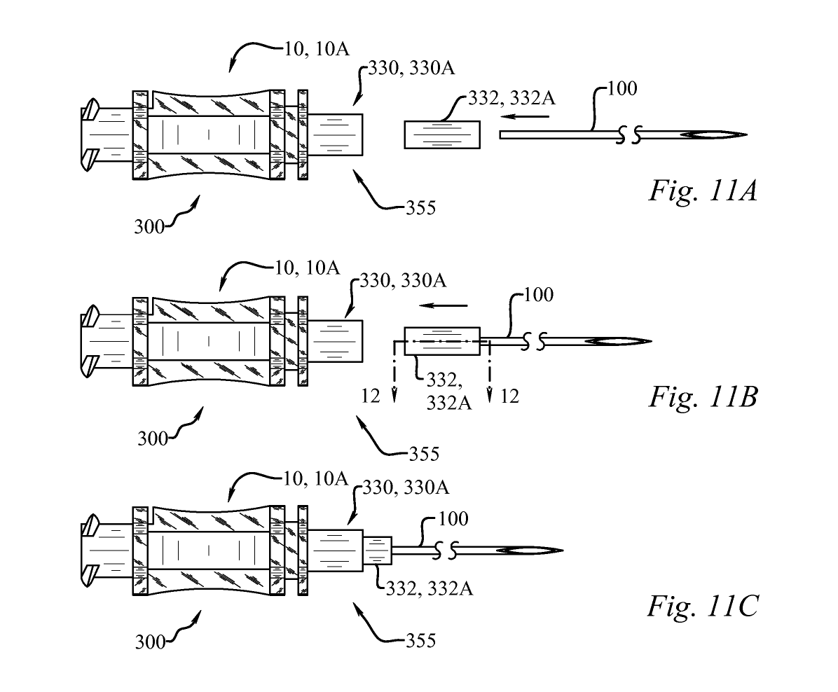

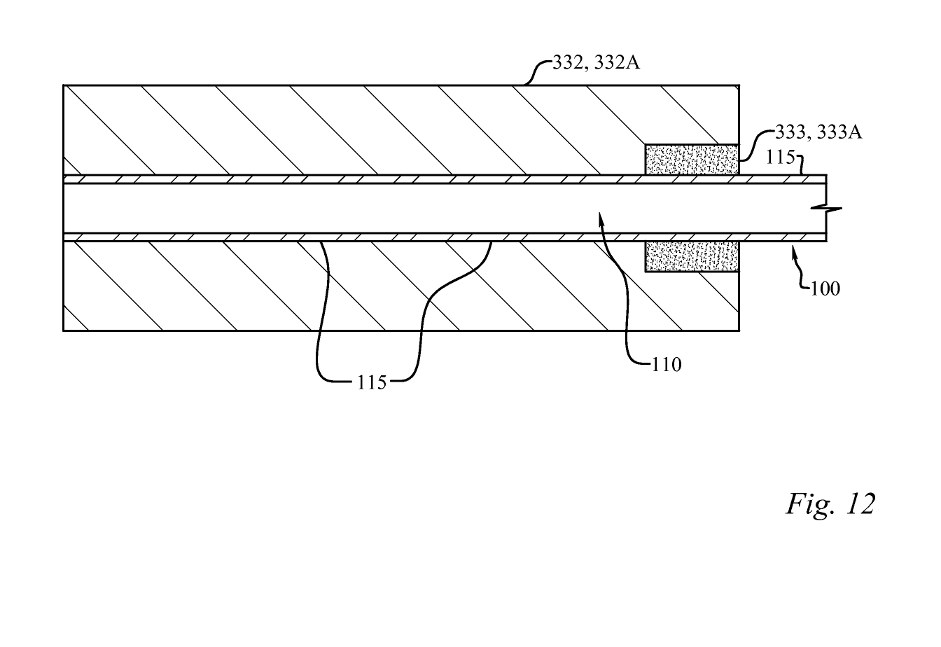

As seen in FIGS. 1-15, in some embodiments, the system of the present invention provides a rotational orientation indicating hollow needle assembly (10) formed generally of three sub-assemblies. The first sub-assembly is a needle (100) having a needle lumen (110) surrounded at least in part by a needle sidewall (115), as seen well in FIGS. 9 and 12. The assembly (10) has a needle proximal end (105) and a needle distal end (155), seen well in FIG. 2. Throughout this specification, the term “proximal” shall mean that portion of the assembly (10) closest to the patient or subject when the assembly (10) is in use, and the term “distal” shall mean that portion of the assembly that is most distant from the patient or subject when the assembly (10) is in use. The assembly has a needle port (120) that places the needle lumen (110) in fluid communication with an external atmosphere, seen well in FIG. 9. Additionally, throughout this specification, the term “bevel” shall also include the side-port hollow needles having needle ports on the needle sidewall (115), such as the “Tuohy” style epidural needle, or various other side-port needle designs. In such cases, the directionality of the term “bevel” shall be equated to the directionality of the side-port needle port (125). The “lumen side” shall refer to the exit port side of any beveled or side port needle.

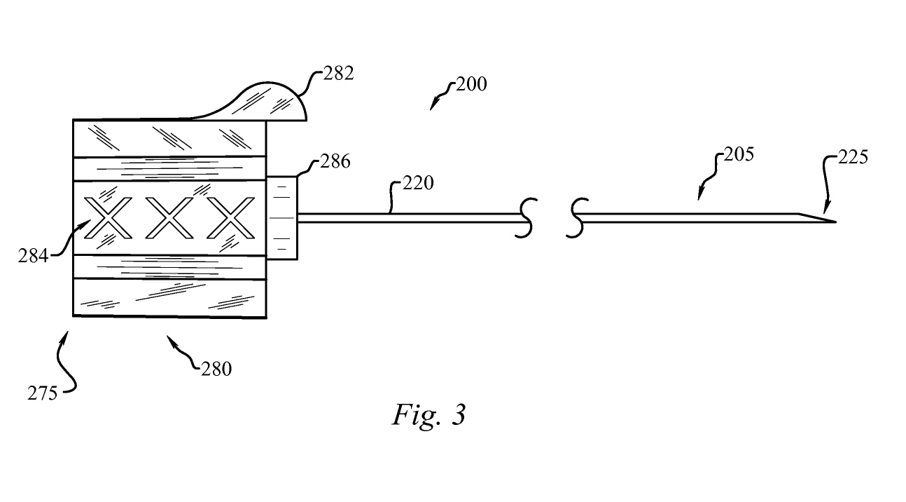

The assembly has a stylet (200), seen well in FIG. 3, substantially reversibly occluding the needle lumen (110) when in an insertion position into a patient or subject. Again with reference to FIG. 3, the stylet (200) includes a stylet shaft (220) with a stylet proximal end (205) and a stylet distal end (275), and a stylet base (280).

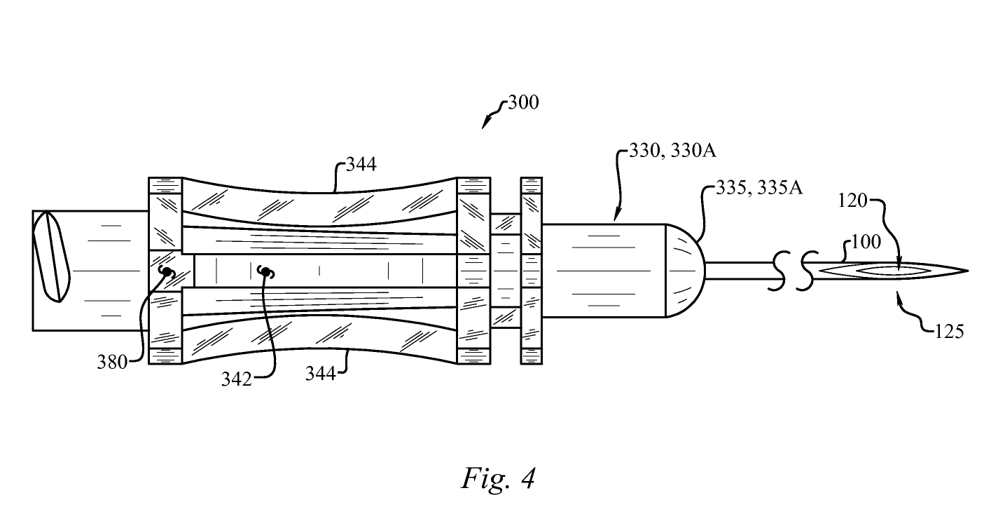

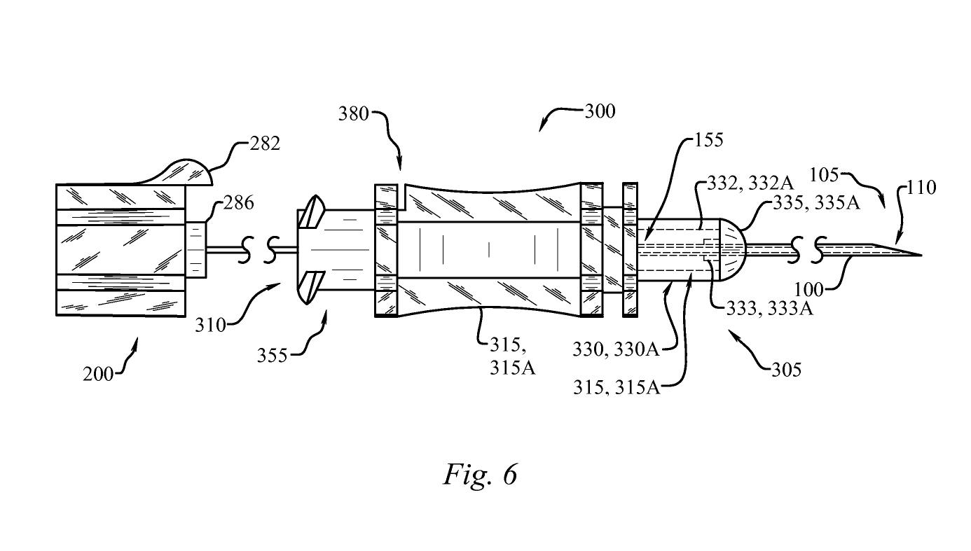

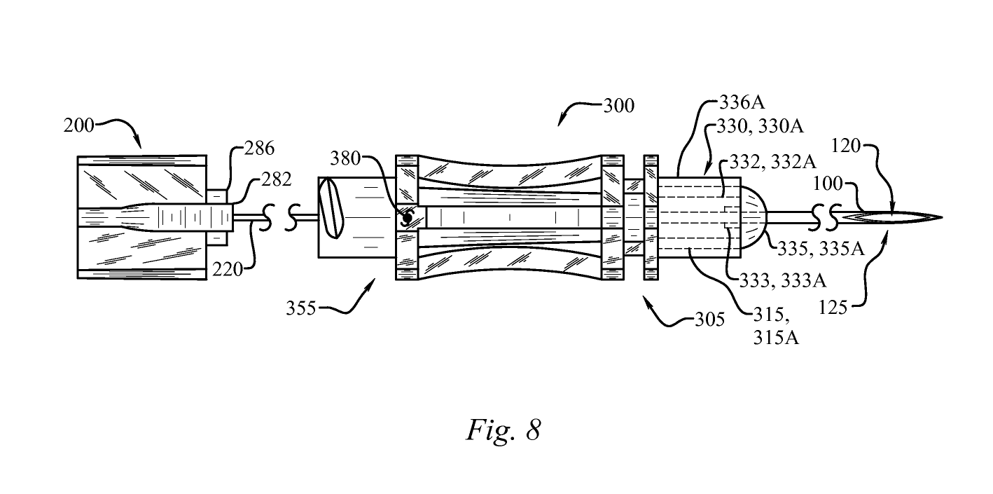

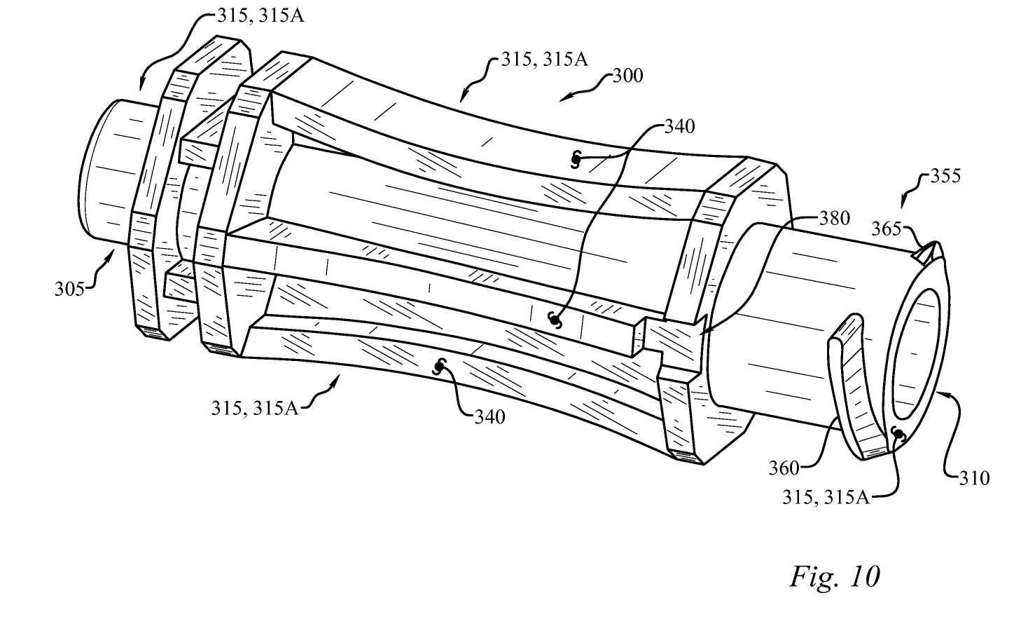

The assembly (10), at its distal end, has a hub (300), seen well in FIGS. 4 and 6, having a hub lumen (310) surrounded at least in part by an at least partially visually clear hub sidewall (315A), connecting a hub proximal end (305) and a hub distal end (355), seen well in FIGS. 6 and 10. By “partially visually clear hub sidewall,” this specification intends that phrase to mean that at least a portion of the hub sidewall (315) is sufficiently clear to human vision so that the presence of materials, such as fluids, and the color and nature of such fluids within the hub lumen (310) may be accurately perceived by a user. The hub proximal end (305) is attached in a predetermined rotational relationship to the needle distal end (155) at a hub-needle attachment (330), seen well in FIG. 8. This places the hub lumen (310) in fluid communication with the needle lumen (110). As seen well in FIG. 10, the at least partially visually clear hub sidewall (315A) may have at least one external surface formed with at least one humanly tactilely discernible surface feature (340) indicating the rotational relationship between the hub (300) and the needle (100).

Thus, by touch alone, an operator grasping the hub (300) may be immediately able to tell, by finger touch alone, the orientation of the needle port (120), and in those embodiments having a needle bevel (125) where the needle port is formed at least partially within the needle bevel (125), the directionality of the needle bevel (125) can likewise be ascertained, as seen well in FIGS. 1, 2, and 9.

Cooperating with the needle (100), the stylet proximal end (205) may formed with a stylet bevel (225), seen well in FIG. 3, cooperating with the needle bevel (125) in an insertion position to form a substantially uniform needle sidewall (115), as seen well in FIG. 9.

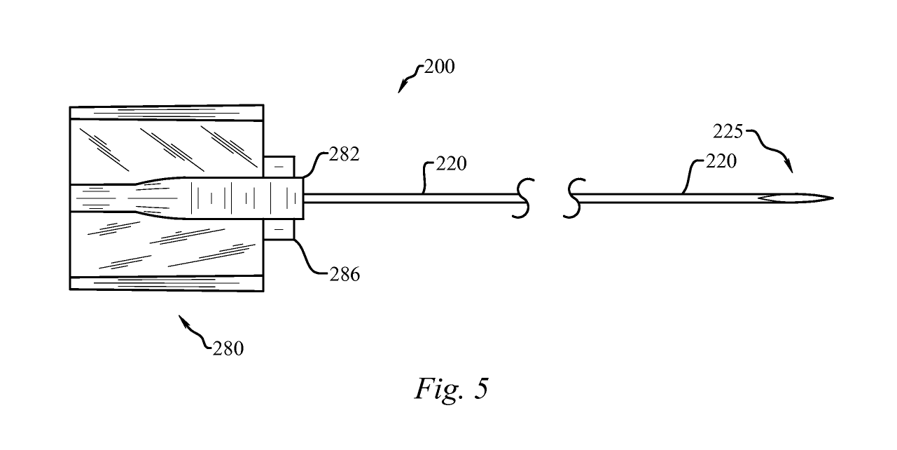

In other embodiments, as also seen well in FIG. 5, the stylet base (280) may be formed with a bevel index tab (282) in a predetermined rotational relationship with the stylet bevel (225) indicating said predetermined rotational relationship. Thus, the stylet (200) may effectively be locked into position when fully inserted into the needle (100), and would not be able to rotate independently of the needle (100).

In various other embodiments, the assembly (10) can include a stylet base (280) that also includes a hub occluder (286) which, as seen by way of example, in FIGS. 5 and 6, when in an insertion position, occludes at least a portion of the hub lumen (310). The hub-needle attachment (330) may be further formed with a needle sleeve (332) surrounding at least a portion of the needle distal end (155) and the needle sleeve (332) may substantially surround at least a portion of the hub lumen (310), as seen well in FIG. 6.

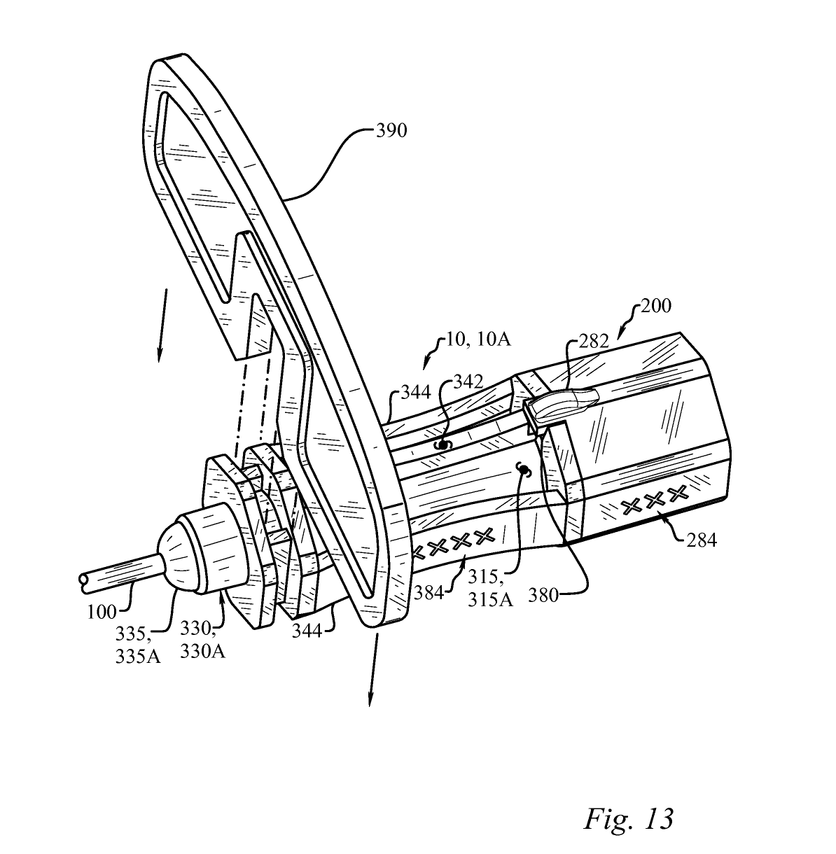

With reference to FIG. 12, the needle sleeve (332) may be adhered at least a portion of the needle distal end (155) with needle sleeve bedding (333) and in another embodiment, at least a portion of the hub-needle attachment (330) may include attachment compound (335), seen well in FIG. 13. Any portion of any of the above detailed assembly (10) components may bear indicia (284, 384), seen well in FIG. 1, and/or may be color-coded, to represent such things as the size (gauge) of the needle, or needle type. Such visually perceptible indicia serve as an additional guide to the humanly tactilely discernible feature (340) and may be of any type perceptible to human vision. These would include colored components comprising all or part of the needle (100), stylet (200), and/or hub (300); as well as visually perceptible indicia of any kind on any portion of the assembly (10). In one particular embodiment at least a portion of the hub-needle attachment (330) comprises a same color as indexed stylet base (280), needle sleeve (332) and attachment compound (335) to allow rapid visual identification and/or confirmation of assembly parameters, including but not limited to size (gauge) of the needle and needle type. It is particularly noted that color indexing does not require that each of the named elements be entirely colored, as it is envisioned that color bands, dots, or other color schemes may be sufficient to allow indexing of the elements.

As described in part above, many variations are possible in forming the humanly tactilely discernible feature (340). All or part of the feature may be an external surface bevel face orientation feature (342) indicating the direction of the needle bevel (125), seen well in FIGS. 1 and 4. Additionally, all or part of the feature (340) may include a bevel orthogonal orientation feature (344) indicating a position orthogonal, or at least a direction other than, to the direction of the needle bevel (125), also seen well in FIGS. 1 and 4. One skilled in the art will realize that there is essentially no limitation upon the nature of the humanly tactile discernible feature (340), other than it be recognizable to the operator and be in a predetermined fixed rotational position to any needle port (120) or needle bevel (125). Furthermore, the spatial relationship between features (340) may be of any type, and the orthogonal arrangement described above is only one of many possible embodiment relationships.

In various embodiments, the hub (300) further may further include a hub-externality connector (360), seen well in FIG. 10. By way of example and not limitation only, this could include a threaded portion suitable for attaching to some external device, again by way of limitation only, such as the well-known “Luer lock connector,” that may be used to connect the assembly to a syringe, tubing, or the like, by means such as a thread (360).

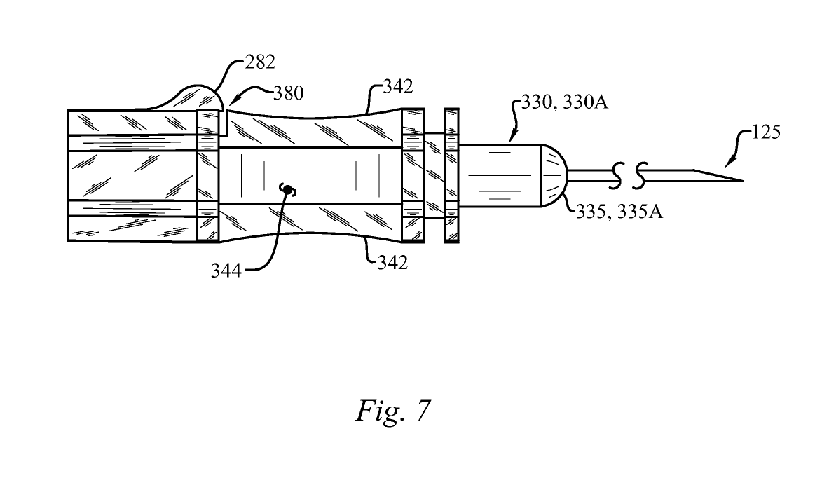

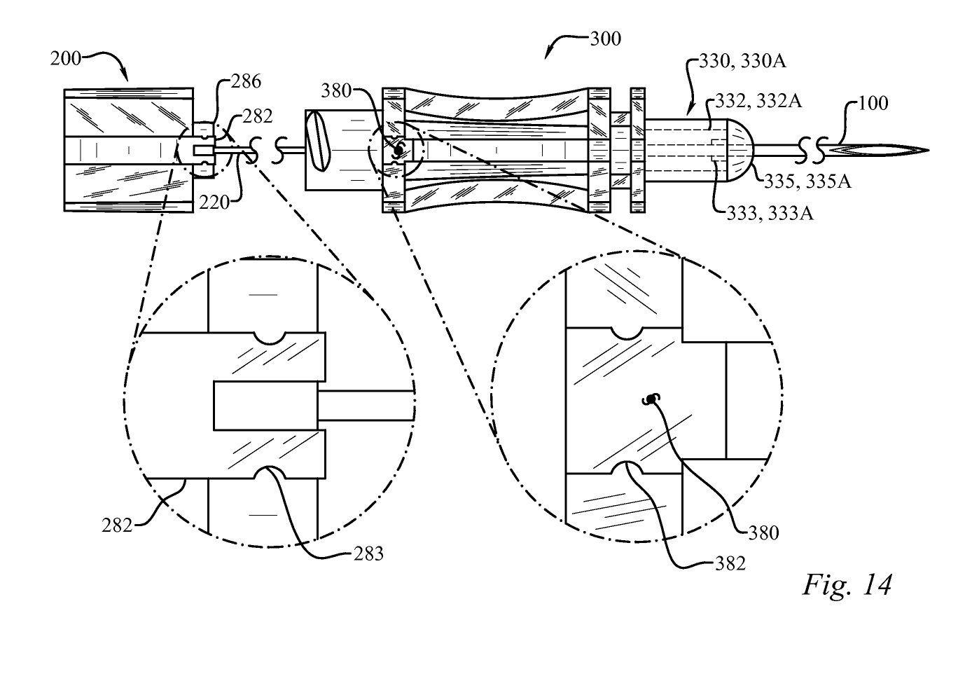

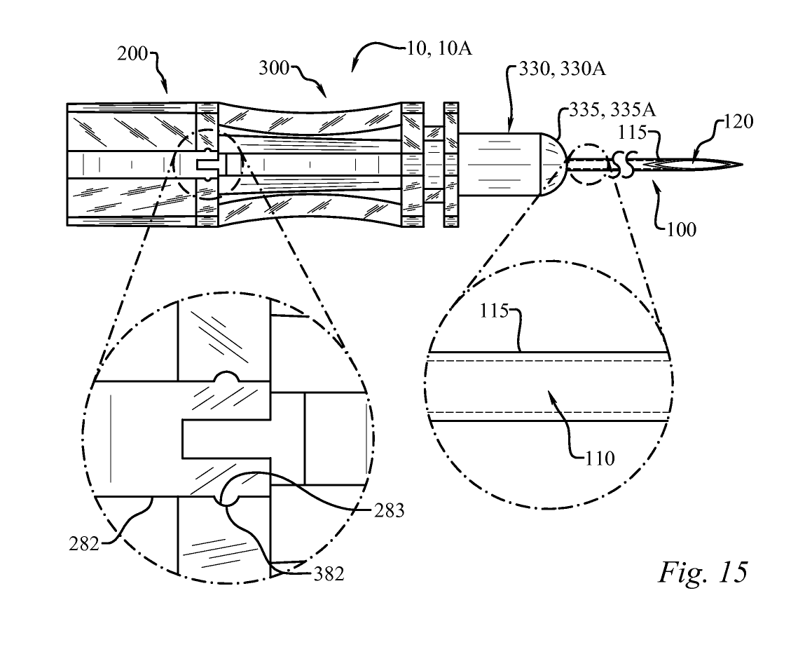

In some embodiments, the bevel index tab (282) cooperates with a bevel index notch (380), seen well in FIGS. 3, 4, and 7, to indicate a predetermined rotational relationship between the needle (100), stylet (200) and hub (300), as seen well in FIGS. 7 and 9. Thusly engaged, and operating in cooperation with the humanly tactile discernible feature (340), the operator may be assured by touch alone, as to the rotational orientation of the entire assembly (10). Such easy tactile discrimination may be aided in other embodiments by the provision of stylet facets (290) that cooperate with a plurality of hub facets (370) indicating a predetermined rotational position between the stylet (200) and the hub (300), as seen in one embodiment in FIG. 1. Thus, the stylet base (280) and hub facets (370) may present a uniform tactile surface to aid in showing proper operational joining. To assist the stylet base (280) in maintaining a firm connection with the bevel index notch (380), the bevel index tab (282) may be configured as a split-tab to create resilience tending to hold pressure against the walls of the bevel index notch (380), as seen in FIGS. 14 and 15. In other embodiments, also seen in FIGS. 14 and 15, a stylet hub retainer (283) may be configured so as to cooperate with a hub-stylet retainer (382) to provide an additional reversible locking action between the stylet (200) and the hub (300). The retainers (283, 382) may be designed in any mutually cooperative manner so as to help secure the stylet (200) and hub (300).

Because of the relatively small size of the assembly (10) in many embodiments, the hub (300) may be provided with a hub wing (390) to make gripping easier, as seen in an exemplary embodiment in FIG. 13. As would be seen by one skilled in the art, the hub wing (390) may be releasable or non-releasable, may be of any convenient shape or size, and may bear any type of visually or tactilely discernible features found useful to users.

In an exemplary embodiment, such as those seen well in FIGS. 1, 9, 10, and 13; but one that is not intended as a limitation of any kind, the needle hub (300) may be configured with two external surface bevel face orientation features (342) in the form of narrow ribs, one rotationally aligned with the needle bevel (125) and one a hundred and eighty degrees radially displaced from the needle bevel (125). In the case where there may be more than one external surface bevel face orientation feature (342), it may be desirable that these features have tactilely separable characteristics.

In some embodiments, the bevel face orientation feature (342) may include a first bevel orientation feature (342) having a first discernible tactile configuration, and a second bevel orientation feature (342) having a second discernible tactile configuration. In such an embodiment, it would be possible to have the first bevel orientation feature (342) identifying a lumen side of the needle bevel (125) and the second bevel orientation feature (342) identifying a side of the needle (100) directly opposite the lumen side of the needle bevel (125). In other embodiments, different tactile configurations of any of the humanly tactilely discernible surface features (340) may identify any particular aspect of the rotational position of a needle (100) as may be desired by a designer.

In some embodiments, there may be two other bevel orthogonal orientation features (344) in the form of wide ribs, one ninety degrees radially displaced from the needle bevel (125) and one two hundred and seventy degrees radially displaced from the needle bevel (125). An operator pinching the hub between a thumb and forefinger thus immediately feels these orientation features (342, 344). When the assembly (10) and thereby the needle bevel (125), is rotated between the thumb and forefinger, these differing orientation features (342, 344) rotate as well, and the difference in tactile sensation immediately alerts the operator to the position of the needle bevel (125).

In a further series of embodiments, also seen illustrated in FIGS. 1-15, a visually size-gauging hollow needle assembly (10A) is taught, which as will be discussed below, may or may not also be rotation orienting. In one embodiment, seen well in FIGS. 1-2, 6-9 and 11-15, a visually size-gauging hollow needle assembly (10A), includes a needle (100) having a needle lumen (110) that is surrounded at least in part by a needle sidewall (115) that connects a needle proximal end (105) a needle distal end (155), and a needle port (120). There is a stylet (200), substantially and reversibly occluding the needle lumen (110) in an insertion position, having a stylet shaft (220) with a stylet proximal end (205), a stylet distal end (275), and a stylet base (280).

The assembly (10A) has a hub (300) having a hub lumen (310) surrounded at least in part by an at least partially visually clear hub sidewall (315A), connecting a hub proximal end (305) and a hub distal end (355). The hub proximal end (305) may be connected to the needle distal end (155) at a visually size-gauging hub-needle attachment (330A) visually indexed to at least one of a plurality of size-gauging needles (100), thereby placing the hub lumen (310) in fluid communication with the needle lumen (110). In at least some embodiments, the visually clear hub sidewall allows a user to both visualize any contents within the hub lumen (310) as well as to clearly see the visually size-gauging hub needle attachment (330A). In a series of embodiments, the visually size-gauging hub-needle attachment (330A) may be of a predetermined color that corresponds to a hollow needle assembly (10A) gauge, or as it might also be known, the hollow needle assembly's (10A) relative needle (100) size or diameter. Thus, in these embodiments, even if a size indicating stylet (200) is removed, or the hollow needle assembly (10A) is separated from its packaging or other gauge indicating indicia, a user may immediately perceive the size or gauge of the needle (100).

The visually size-gauging hub-needle attachment (330A) may have a needle sleeve (332), seen well in FIGS. 2, 6, 8 and 11, surrounding at least a portion of the needle distal end (155) and the needle sleeve (332) may substantially surrounded by at least a portion of the hub lumen (310).

In a further series of embodiments, also seen well in FIGS. 2, 6, 8 and 11, the visually size-gauging hub-needle attachment (330A) may be further formed with a visually size-gauging needle sleeve (332A) surrounding at least a portion of the needle distal end (155) and the visually size-gauging needle sleeve (332A) may be substantially surrounded by at least a portion of the hub lumen (310). Again with reference to FIGS. 2, 6, 8 and 11, as well as FIG. 12, the needle sleeve (332) may be adhered to at least a portion of the needle distal end (155) with a needle sleeve bedding (333), and such needle sleeve bedding (333) may be a visually size-gauging needle sleeve bedding (333A). In some embodiments, the visually size-gauging needle sleeve (332A) may be adhered to at least a portion of the needle distal end (155) with a needle sleeve bedding (333), and such needle sleeve bedding (333) may be a visually size-gauging needle sleeve bedding (333A).

In another series of embodiments, seen well in FIGS. 1-2, 4, 6-9 and 13-15, the visually size-gauging hollow needle assembly (10A) may have a portion of the visually size-gauging hub-needle attachment (330A) including an attachment compound (335), and such attachment compound (335) may be a visually size-gauging attachment compound (335A).

In yet another series of embodiments, the visually size-gauging hollow needle assembly (10A) may have external visually-gauging needle indicia (336A) placed on the external surface of either the at least partially visually clear hub sidewall (315A) and/or the needle sleeve (332). In a common series of embodiments, seen well in FIGS. 6, 8 and 9, such external visually size-gauging needle indicia (336A) may be an external band that is placed around the at least partially visually clear hub (315A) and/or the needle sleeve (332). By way of example and not limitation, FIG. 6 shows an area of at least partially visually clear hub sidewall (315A) overlying a visually size-gauging needle sleeve (332A) which may be seen through the at least partially visually clear hub sidewall (315A). In such an embodiment, external visually size-gauging needle indicia (336A) may be used, but may not be required, in those embodiments where a visually size-gauging needle sleeve (332A) is visible through the at least partially visually clear hub sidewall (315A). FIG. 8 shows an area of external visually size-gauging needle indicia (336A) overlying an area of at least partially visually clear hub sidewall (315A), which in turn overlays a needle sleeve (332) or a visually size-gauging needle sleeve (332A). In such an embodiment, either a needle sleeve (332) or a visually size-gauging needle sleeve (332A) may be utilized, as the visually-size-gauging needle indicia (336A) lies outside of the needle sleeve (332) or visually size-gauging needle sleeve (332A). FIG. 9 shows an area of external visually size-gauging needle indicia (336) directly overlying a needle sleeve (332) or visually size-gauging needle sleeve (332A) without an intervening layer of at least partially visually clear hub sidewall (315A). Again, in such an embodiment, either a needle sleeve (332) or a visually size-gauging needle sleeve (332A) may be utilized, as the visually-size-gauging needle indicia (336) lies outside of the needle sleeve (332) or visually size-gauging needle sleeve (332A). However, it is expressly intended that any visually-size gauging needle indicia (336A) that is placed anywhere on the hollow needle assembly (10A) surface would suffice, the only requirement being that in some manner, the hollow needle assembly gauge (10A) be indicated by visually perceptible means.

In each of the embodiments using one or more of a visually size-gauging needle sleeve (332A), a visually size-gauging needle sleeve bedding (333A), a visually size-gauging attachment compound (335A), and/or external visually-gauging needle indicia (336A), a common series of embodiments would use a predetermined series of colors to indicate relative needle (100) gauge, but it is expressly intended by the specification that any visually perceptible method of visually size gauging the hollow needle assembly (10A) would be possible.

In many versions of the visually size-gauging hollow needle assembly (10A) the needle proximal end (105) may have a needle bevel (125) and the needle port (120) may be at least partially within the needle bevel (125), as seen well in FIGS. 1, 2, and 4, among others. The needle proximal end (105) may also be made with a needle port (120) formed at least partially within the needle sidewall (115).

The needle (100) cooperates with the stylet (200) in various ways. In some embodiments, the stylet proximal end (205) is formed with a stylet bevel (225), seen in FIG. 3, cooperating with the needle bevel (125) in an insertion position to form a substantially concentric uniform needle sidewall (115), as seen in FIGS. 9 and 12. As seen well in FIGS. 8 and 9, the stylet base (280) may formed with a bevel index tab (282) in a predetermined rotational relationship with the stylet bevel (225) indicating said predetermined rotational relationship.

Just as with other embodiments of rotational orientation indicating hollow needle assemblies (10) previously described, the visually size-gauging hollow needle assembly (10A), as seen well in FIG. 2, may have an at least partially visually clear hub sidewall (315A) with at least one external surface formed with at least one humanly tactilely discernible humanly tactilely discernible feature (340), in order to indicate a predetermined rotational relationship between the hub (300) and the needle (100).

Such a humanly tactilely discernible humanly tactilely discernible feature (340) may be an external surface bevel face orientation feature (342) indicating the direction of a needle bevel (125). In one particular embodiment, seen well in FIG. 4, the bevel face orientation feature (342) has a first bevel orientation feature (342) having a first discernible tactile configuration, and a second bevel orientation feature (342) having a second discernible tactile configuration. In such an embodiment, the first bevel orientation feature (342) may identify a lumen side of the needle bevel (125) and the second bevel orientation feature (342) may identify a side of the needle (100) directly opposite the lumen side of the needle bevel (125).

In various embodiments, the humanly tactilely discernible feature (340) is a bevel orthogonal orientation feature (344) indicating a position orthogonal to the direction of the needle bevel (125). In other embodiments, the bevel index tab (282) cooperates with a bevel index notch (380) to indicate a predetermined rotational relationship between the needle (100), stylet (200) and hub (300). A plurality of stylet facets (290) may cooperate with a plurality of hub facets (370) to indicate a predetermined rotational relationship between the stylet (200) and the hub (300).

To allow for visual identification of the gauge of a hollow needle assembly (10A), the hollow needle assembly (10A) may have a visually size-gauging hub-needle attachment (330A) that can include a visually size-gauging hub-needle attachment (330A) component selected from the group of visually size-gauging hub-needle attachment (330A) components consisting of a visually size-gauging needle sleeve (332A), visually size-gauging needle sleeve bedding (333A), visually size-gauging attachment compound (335A), external visually size-gauging indicia (336A), and combinations thereof. In particular, but not exclusively, the visually size-gauging hub-needle attachment (330A) component may be visually size-gauging by means of a predetermined visually perceptible color. However, as before, it is specifically intended that any visually perceptual means of differentiation is possible.

In one specific embodiment, the visually size-gauging hollow needle assembly (10A) may have a needle (100) having a needle lumen (110) surrounded at least in part by a needle sidewall (115) connecting a needle proximal end (105) formed with a needle bevel (125), as well as a needle distal end (155), and a needle port (120) at least partially within the needle bevel (125). There may be a stylet (200), substantially reversibly occluding the needle lumen (110) in an insertion position, that has a stylet shaft (220) with a stylet proximal end (205), a stylet distal end (275), and a stylet base (280). In such specific embodiments, there may be a hub (300) having a hub lumen (310) surrounded at least in part by an at least partially visually clear hub sidewall (315A), connecting a hub proximal end (305) and a hub distal end (355). The hub proximal end (305) may be attached in a predetermined rotational relationship to the needle distal end (155) at a visually size-gauging hub-needle attachment (330A), thereby placing the hub lumen (310) in fluid communication with the needle lumen (110). The at least partially visually clear hub sidewall (315A) may have at least one external surface formed with at least one humanly tactilely discernible surface feature (340) including an external surface bevel face orientation feature (342). This may indicate the direction of the needle bevel (125) and the bevel face orientation feature (342) may have a first bevel orientation feature (342) having a first discernible tactile configuration and a second bevel orientation feature (342) having a second discernible tactile configuration. The first bevel orientation feature (342) may identify a lumen side of the needle bevel (125) and the second bevel orientation feature (342) may identify a side of the needle (100) directly opposite the lumen side of the needle bevel (125), thus indicating the rotational relationship between the hub (300) and the needle (100).

In another specific embodiment, the visually size-gauging hollow needle assembly (10A) also has a needle (100) having a needle lumen (110) surrounded at least in part by a needle sidewall (115) connecting a needle proximal end (105) formed with a needle bevel (125). The needle may have a needle distal end (155), and a needle port (120) at least partially within the needle bevel (125). There may be a stylet (200), substantially reversibly occluding the needle lumen (110) in an insertion position, having a stylet shaft (220) with a stylet proximal end (205), a stylet distal end (275), and a stylet base (280). In such a specific embodiment, there may be a hub (300) having a hub lumen (310) surrounded at least in part by an at least partially visually clear hub sidewall (315A), connecting a hub proximal end (305) and a hub distal end (355). The hub proximal end (305) may be attached in a predetermined rotational relationship to the needle distal end (155) at a visually size-gauging hub-needle attachment (330A) having a visually size-gauging needle sleeve (332A) surrounding at least a portion of the needle distal end (155). The visually size-gauging needle sleeve (332A) may be substantially surrounded by at least a portion of the hub lumen (310), thereby placing the hub lumen (310) in fluid communication with the needle lumen (110). At least a portion of the hub-needle attachment (330) may include a visually size-gauging attachment compound (335) and the at least partially visually clear hub sidewall (315A) may have at least one external surface formed with at least one humanly tactilely discernible surface feature (340) having an external surface bevel face orientation feature (342) indicating the direction of the needle bevel (125). The bevel face orientation feature (342) may consist of a first bevel orientation feature (342) having a first discernible tactile configuration, and a second bevel orientation feature (342) having a second discernible tactile configuration. The first bevel orientation feature (342) may identify a lumen side of the needle bevel (125) and the second bevel orientation feature (342) may identify a side of the needle (100) directly opposite the lumen side of the needle bevel (125), indicating the rotational relationship between the hub (300) and the needle (100).

Numerous alterations, modifications, and variations of the preferred embodiments disclosed herein will be apparent to those skilled in the art and they are all anticipated and contemplated to be within the spirit and scope of the instant invention. For example, although specific embodiments have been described in detail, those with skill in the art will understand that the preceding embodiments and variations can be modified to incorporate various types of substitute and or additional or alternative manufacturing processes and materials, relative arrangement of elements, and dimensional configurations. Accordingly, even though only few variations of the present invention are described herein, it is to be understood that the practice of such additional modifications and variations and the equivalents thereof, are within the spirit and scope of the invention as defined in the following claims. The corresponding structures, materials, acts, and equivalents of all means or step plus function elements in the claims below are intended to include any structure, material, or acts for performing the functions in combination with other claimed elements as specifically claimed.

I claim:

1. A visually size-gauging hollow needle assembly (10A), comprising;
a needle (100) having a needle lumen (110) surrounded at least in part by a needle sidewall (115) connecting a needle proximal end (105) a needle distal end (155), and a needle port (120);
a stylet (200), substantially reversibly occluding the needle lumen (110) in an insertion position, having a stylet shaft (220) with a stylet proximal end (205), a stylet distal end (275), and a stylet base (280); and
a hub (300) having a hub lumen (310) surrounded at least in part by an at least partially visually clear hub sidewall (315A), connecting a hub proximal end (305) and a hub distal end (355), wherein the hub proximal end (305) is connected to the needle distal end (155) at a visually size-gauging hub-needle attachment (330A) visually indexed to at least one of a plurality of size-gauging needles (100), thereby placing the hub lumen (310) in fluid communication with the needle lumen (110).

2. The visually size-gauging hollow needle assembly (10A) according to claim 1, wherein the visually size-gauging hub-needle attachment (330A) is further formed with a needle sleeve (332) surrounding at least a portion of the needle distal end (155) and the needle sleeve (332) is substantially surrounded by at least a portion of the hub lumen (310).

3. The visually size-gauging hollow needle assembly (10A) according to claim 1, wherein the visually size-gauging hub-needle attachment (330A) is further formed with a visually size-gauging needle sleeve (332A) surrounding at least a portion of the needle distal end (155) and the visually size-gauging needle sleeve (332A) is substantially surrounded by at least a portion of the hub lumen (310).

4. The visually size-gauging hollow needle assembly (10A) according to claim 2, wherein the needle sleeve (332) is adhered to at least a portion of the needle distal end (155) with a needle sleeve bedding (333).

5. The visually size-gauging hollow needle assembly (10A) according to claim 2, wherein the needle sleeve (332) is adhered to at least a portion of the needle distal end (155) with a visually size-gauging needle sleeve bedding (333A).

6. The visually size-gauging hollow needle assembly (10A) according to claim 3, wherein the visually size-gauging needle sleeve (332A) is adhered to at least a portion of the needle distal end (155) with a needle sleeve bedding (333).

7. The visually size-gauging hollow needle assembly (10A) according to claim 3, wherein the visually size-gauging needle sleeve (332A) is adhered to at least a portion of the needle distal end (155) with a visually size-gauging needle sleeve bedding (333A).

8. The visually size-gauging hollow needle assembly (10A) according to claim 1, wherein at least a portion of the visually size-gauging hub-needle attachment (330A) comprises an attachment compound (335).

9. The visually size-gauging hollow needle assembly (10A) according to claim 1, wherein at least a portion of the visually size-gauging hub-needle attachment (330A) comprises a visually size-gauging attachment compound (335A).

10. The visually size-gauging hollow needle assembly (10A) according to claim 2, wherein a structure selected from the group of structures consisting of the at least partially visually clear hub sidewall (315A) and the needle sleeve (332) and combinations thereof bears external visually size-gauging needle indicia (336A).

11. The visually size-gauging hollow needle assembly (10A) according to claim 1, wherein the needle proximal end (105) is formed with a needle bevel (125) and the needle port (120) is at least partially within the needle bevel (125).

12. The visually size-gauging hollow needle assembly (10A) according to claim 11, wherein the stylet proximal end (205) is formed with a stylet bevel (225) cooperating with the needle bevel (125) in an insertion position to form a substantially concentric uniform needle sidewall (115).

13. The visually size-gauging hollow needle assembly (10A) according to claim 12, wherein the stylet base (280) is formed with a bevel index tab (282) in a predetermined rotational relationship with the stylet bevel (225) indicating said predetermined rotational relationship.

14. The visually size-gauging hollow needle assembly (10A) according to claim 1, wherein the at least partially visually clear hub sidewall (315A) has at least one external surface formed with at least one humanly tactilely discernible humanly tactilely discernible feature (340) indicating a predetermined rotational relationship between the hub (300) and the needle (100).

15. The visually size-gauging hollow needle assembly (10A) according to claim 14, wherein the at least one humanly tactilely discernible humanly tactilely discernible feature (340) is an external surface bevel face orientation feature (342) indicating the direction of a needle bevel (125).

16. The visually size-gauging hollow needle assembly (10A) according to claim 15, wherein the bevel face orientation feature (342) further comprises a first bevel orientation feature (342) having a first discernible tactile configuration, and a second bevel orientation feature (342) having a second discernible tactile configuration, and wherein the first bevel orientation feature (342) identifies a lumen side of the needle bevel (125) and the second bevel orientation feature (342) identifies a side of the needle (100) directly opposite the lumen side of the needle bevel (125).

17. The visually size-gauging hollow needle assembly (10A) according to claim 14, wherein the humanly tactilely discernible feature (340) is a bevel orthogonal orientation feature (344) indicating a position orthogonal to the direction of the needle bevel (125).

18. The visually size-gauging hollow needle assembly (10A) according to claim 13, wherein the bevel index tab (282) cooperates with a bevel index notch (380) to indicate a predetermined rotational relationship between the needle (100), stylet (200) and hub (300).

19. The visually size-gauging hollow needle assembly (10A) according to claim 1, wherein a plurality of stylet facets (290) cooperate with a plurality of hub facets (370) indicating a predetermined rotational relationship between the stylet (200) and the hub (300).

20. The visually size-gauging hollow needle assembly (10A) according to claim 1, wherein the visually size-gauging hub-needle attachment (330A) further comprises a visually size-gauging hub-needle attachment (330A) component selected from the group of visually size-gauging hub-needle attachment (330A) components consisting of a visually size-gauging needle sleeve (332A), visually size-gauging needle sleeve bedding (333A), visually size-gauging attachment compound (335A), external visually size-gauging indicia (336A) and combinations thereof.

21. The visually size-gauging hollow needle assembly (10A) according to claim 20, wherein the visually size-gauging hub-needle attachment (330A) component is visually size-gauging by means of a predetermined visually perceptible color.

22. A visually size-gauging hollow needle assembly (10A), comprising;
a needle (100) having a needle lumen (110) surrounded at least in part by a needle sidewall (115) connecting a needle proximal end (105) formed with a needle bevel (125), a needle distal end (155), and a needle port (120) at least partially within the needle bevel (125);
a stylet (200), substantially reversibly occluding the needle lumen (110) in an insertion position, having a stylet shaft (220) with a stylet proximal end (205), a stylet distal end (275), and a stylet base (280); and
a hub (300) having a hub lumen (310) surrounded at least in part by an at least partially visually clear hub sidewall (315A), connecting a hub proximal end (305) and a hub distal end (355), wherein the hub proximal end (305) is attached in a predetermined rotational relationship to the needle distal end (155) at a visually size-gauging hub-needle attachment (330A), thereby placing the hub lumen (310) in fluid communication with the needle lumen (110), and the at least partially visually clear hub sidewall (315A) has at least one external surface formed with at least one humanly tactilely discernible surface feature (340) further comprising an external surface bevel face orientation feature (342) indicating the direction of the needle bevel (125) and wherein the bevel face orientation feature (342) further comprises a first bevel orientation feature (342) having a first discernible tactile configuration, and a second bevel orientation feature (342) having a second discernible tactile configuration, and wherein the first bevel orientation feature (342) identifies a lumen side of the needle bevel (125) and the second bevel orientation feature (342) identifies a side of the needle (100) directly opposite the lumen side of the needle bevel (125), indicating the rotational relationship between the hub (300) and the needle (100).

23. A visually size-gauging hollow needle assembly (10A), comprising;
a needle (100) having a needle lumen (110) surrounded at least in part by a needle sidewall (115) connecting a needle proximal end (105) formed with a needle bevel (125), a needle distal end (155), and a needle port (120) at least partially within the needle bevel (125);
a stylet (200), substantially reversibly occluding the needle lumen (110) in an insertion position, having a stylet shaft (220) with a stylet proximal end (205), a stylet distal end (275), and a stylet base (280); and
a hub (300) having a hub lumen (310) surrounded at least in part by an at least partially visually clear hub sidewall (315A), connecting a hub proximal end (305) and a hub distal end (355), wherein the hub proximal end (305) is attached in a predetermined rotational relationship to the needle distal end (155) at a visually size-gauging hub-needle attachment (330A) having a visually size-gauging needle sleeve (332A) surrounding at least a portion of the needle distal end (155) and the visually size-gauging needle sleeve (332A) is substantially surrounded by at least a portion of the hub lumen (310), thereby placing the hub lumen (310) in fluid communication with the needle lumen (110) and at least a portion of the hub-needle attachment (330) comprises a visually size-gauging attachment compound (335) and the at least partially visually clear hub sidewall (315A) has at least one external surface formed with at least one humanly tactilely discernible surface feature (340) further comprising an external surface bevel face orientation feature (342) indicating the direction of the needle bevel (125) and wherein the bevel face orientation feature (342) further comprises a first bevel orientation feature (342) having a first discernible tactile configuration, and a second bevel orientation feature (342) having a second discernible tactile configuration, and wherein the first bevel orientation feature (342) identifies a lumen side of the needle bevel (125) and the second bevel orientation feature (342) identifies a side of the needle (100) directly opposite the lumen side of the needle bevel (125), indicating the rotational relationship between the hub (300) and the needle (100).
